SEQUENCE LISTING (1 sequence)

SEQ ID NO: 1 - DNA, 870 bp, Homo sapiens
ggtgacagctgatgtgtattgggctcttactgtcagccgtattttatgccatgctctgcaaaccagcgaggccggcgctgcagacccattactcagacgggaacagagaggccgggagaagcgaaatcacccaggggctggggtcgtcgcagccaggagagactccggccctcaccaccacctgggcgagatcacgctgcaaacggggccccttcccggtgcagcccctccacccccagcagaacttgggaaaggcgcggtccgggactctccgcggatcgggaggggattccaggcccccccgaaagtccgggccgcctcgcgcgctggaaatcccgcgcgcgccccgaaccgcggctcggctgccgggaaatcaggagaaaaaaacttctgcttttttttcttttctggcattcgcggtcacctacccggcccccgcgcgccctcctcccggttctcgcccccacgtggggcgcccccgcacgccgctcctccccctcccctccgtcggccaaccgcagagctagctgcactcgcccttgtctttccaccaataggaggggcgaatgactccactgaggccacgcccaatgttcaagtctataaaagtcggtgccggaggctcccagctcagatcgccgaagcgtcggactaccgttggtttccgcaacttcctggattatcctcgccaaggactttgcaatatatttttccgccttttctggaaggatttcgctgcttcccgaaggtcttggacgagcgctctagctctgtgggaaggttttgggctctctggctcggattttgcaatttctccctggggactgccgtggagccgcatccactgtggattataattgcaacatgacg

(19) United States
(12) Patent Application Publication
(10) Pub. No.: US 2004/0101916 A1
(43) Pub. Date: May 27, 2004

(54) Treatment of liver diseases

(76) Inventor: Yun Yen, Arcadia, CA (US)

Correspondence Address: FISH & RICHARDSON PC, 225 FRANKLIN ST, BOSTON, MA, 02110, US

(21) Appl. No.: 10/640,884

(22) Filed: Aug. 14, 2003

Related U.S. Application Data
(60) Provisional application No. 60/404,512, filed on Aug. 19, 2002.

(51) Int. Cl. G01N 33/574

(52) U.S. Cl. 435/7.23

(57) ABSTRACT

A method of determining whether a subject is suffering from or at risk for developing a liver disease. The method includes providing a sample from the subject and determining a GADD45β expression level or a GADD45β activity level in the sample. If the GADD45β expression level or the GADD45β activity level in the sample is lower than that in a sample from a normal subject, it indicates that the subject is suffering from or at risk for developing a liver disease. Also disclosed are a method of identifying a compound for treating a liver disease and a method of treating such a disease.

### CROSS REFERENCE TO RELATED APPLICATIONS

Pursuant to 35 USC § 119(e), this application claims the benefit of prior U.S. provisional application 60/404,512, filed Aug. 19, 2002.

### BACKGROUND

Cirrhosis has been viewed as a precursor of liver cancer. There is strong epidemiologic evidence suggesting that cirrhosis and liver cancer are associated with alcohol consumption. Once cirrhosis has occurred, cessation of drinking does not prevent development of liver cancer.

### SUMMARY

This invention relates to the use of a growth arrest and DNA-damage-inducible gene 45 beta (GADD45β) in diagnosing and treating liver diseases (e.g., cirrhosis and liver cancer), and in identifying therapeutic compounds for treating such diseases.

In one aspect, this invention features a method of determining whether a subject is suffering from or at risk for developing a liver disease, e.g., cirrhosis or liver cancer. In one example, the method includes providing a sample (e.g., a liver sample) from a subject and determining the GADD45β expression level in the sample. If the GADD45β expression level in the sample is lower than that in a sample from a normal subject, it indicates that the subject is suffering from or at risk for developing a liver disease. The GADD45β expression level can be determined by measuring the amount of the GADD45β mRNA, or the GADD45β protein. The GADD45β mRNA level can be determined, for example, by in situ hybridization, PCR, or Northern blot analysis. The GADD45β protein level can be determined, for example, by Western blot analysis. In another example, the method includes providing a sample from a subject and determining the GADD45β activity level in the sample. If the GADD45β activity level in the sample is lower than that in a sample from a normal subject, it indicates that the subject is suffering from or at risk for developing a liver disease. The GADD45β activity can be determined, e.g., by measuring its ability to inhibit cell growth and to induce apoptotic cell death in abnormal liver cells.

In another aspect, this invention features a method of identifying a compound for treating a liver disease, e.g., cirrhosis or liver cancer. In one example, the method includes contacting a compound with a cell (e.g., a liver cell) and determining the GADD45β expression level in the cell. If the GADD45β expression level in the presence of the compound is higher than that in the absence of the compound, the compound is a candidate for treating a liver disease. The cell can be a cell treated with S-adenosylmethionine or an alcohol, a cell expressing a p53 gene at a level lower than that in a normal cell, a cell in which transcription of the GADD45β gene is directed by a promoter containing SEQ ID NO: 1 and that expresses a CCAAT/NF-Y factor or an NF-κB factor, or a cell of a combination of just-described characteristics. In another example, the method includes contacting a compound with a cell and determining a GADD45β activity level in the cell. If the GADD45β activity level in the presence of the compound is higher than that in the absence of the compound, the compound is a candidate for treating a liver disease.

SEQ ID NO:1 is nucleotides −870 through −1 of the GADD45β gene:

(SEQ ID NO:1)

ggtgacagct gatgtgtatt gggctcttac tgtcagccgt
attttatgcc atgctctgca aaccagcgag gccggcgctg
cagacccatt actcagacgg gaacagagag qccgggagaa
gcgaaatcac ccaggggctg gggtcgtcgc agccaggaga
gactccggcc ctcaccacca cctgggcgag atcacgctgc
aaacggggcc ccttcccggt gcagcccctc cacccccagc
agaacttggg aaaggcgcgg tccgggactc tccgcggatc
gggaggggat tccaggcccc cccgaaagtc cgggccgcct
cgcgcgctgg aaatcccgcg cgcgccccga accgcggctc
ggctgccggg aaatcaggag aaaaaaactt ctgctttttt
ttcttttctg gcattcgcgg tcacctaccc ggcccccgcg
cgccctcctc ccggttctcg cccccacgtg gggcgccccc
gcacgccgct cctccccctc ccctccgtcg gccaaccgca
gagctagctg cactcgccct tgtctttcca ccaataggag
gggcgaatga ctccactgag gccacgccca atgttcaagt
ctataaaagt cggtgccgga ggctcccagc tcagatcgcc
gaagcgtcgg actaccgttg gtttccgcaa cttcctggat
tatcctcgcc aaggactttg caatatattt ttccgccttt
tctggaagga tttcgctgct tcccgaaggt cttggacgag
cgctctagct ctgtgggaag gttttgggct ctctggctcg
gattttgcaa tttctccctg gggactgccg tggagccgca
tccactgtgg attataattg caacatgacg

In still another aspect, this invention features a method of treating a liver disease, e.g., cirrhosis or liver cancer. The method includes identifying a subject suffering from or being at risk for developing a liver disease and administering to the subject a composition to increase the GADD45β level in the subject. The composition can contain a nucleic acid encoding a GADD45β protein, a nucleic acid encoding a p53 protein, a GADD45β protein, a p53 protein, S-adenosylmethionine, or a combination thereof. A “GADD45β protein” or a “p53 protein” refers to both a wild-type GADD45β protein or a wild-type p53 protein and its variants with an equivalent biological function (e.g., a fragment of a wild-type GADD45β protein or a wild-type p53 protein). The composition can be administered directly to a liver cell in the subject.

Also within the scope of this invention is a pharmaceutical composition for treating a liver disease, e.g., cirrhosis or liver cancer. The composition can contain a nucleic acid encoding a GADD45β protein and a pharmaceutically acceptable carrier. It can also contain a GADD45β protein itself and a pharmaceutically acceptable carrier.

The present invention provides methods for diagnosing and treating a liver disease associated with insufficient expression of the GADD45β gene. The details of one or more embodiments of the invention are set forth in the accompanying description below. Other features, objects, and advantages of the invention will be apparent from the detailed description, and from the claims.

### DETAILED DESCRIPTION

The present invention is based on an unexpected discovery that the GADD45β gene is down-regulated in human cirrhosis and liver cancer, and the expression of the GADD45β gene is induced by S-Adenosylmethionine (SAMe) and is p53-dependent.

This invention provides methods for diagnosing and treating liver diseases (e.g., cirrhosis and liver cancer), and identifying therapeutic compounds for treating such diseases.

A diagnostic method of this invention involves comparing the GADD45β gene expression level or the GADD45β protein activity level in a sample (e.g., a liver sample) prepared from a subject with that in a sample prepared from a normal person, i.e., a person who does not suffer from a liver disease. A lower GADD45β expression or activity level indicates that the subject is suffering from or at risk for developing a liver disease. The methods of this invention can be used on their own or in conjunction with other procedures to diagnose liver diseases in appropriate subjects.

The GADD45β expression level can be determined at either the mRNA level or at the protein level. Methods of measuring mRNA levels in a tissue sample are known in the art. In order to measure mRNA levels, cells can be lysed and the levels of GADD45β mRNA in the lysates or in RNA purified or semi-purified from the lysates can be determined by any of a variety of methods including, without limitation, hybridization assays using detectably labeled GADD45β -specific DNA or RNA probes and quantitative or semi-quantitative RT-PCR methodologies using appropriate GADD45β -specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

Methods of measuring protein levels in a tissue sample are also known in the art. Many such methods employ antibodies (e.g., monoclonal or polyclonal antibodies) that bind specifically to a GADD45β protein. In such assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to lysates of cells, and others (e.g., immunohistological methods or fluorescence flow cytometry) applied to histological sections or unlysed cell suspensions. Methods of measuring the amount of label will be depend on the nature of the label and are well known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

The GADD45β activity can be determined by methods well known in the art, e.g., by measuring its ability to inhibit cell growth and to induce apoptotic cell death in abnormal liver cells (Takekawa and Saito (1998) Cell 85, 521-530; Nakayama, et al. (1999) Biol. Chem. 274, 24766-24772; Selvakumaran, et al. (1994) Mol. Cell Biol. 14, 2352-2360; and Liebermann and Hoffman (1998) Oncogene 17, 3319-3329).

This invention also provides a method for identifying candidate compounds (e.g., proteins, peptides, peptidomimetics, peptoids, antibodies, or small molecules) that increase the GADD45β gene expression level or GADD45β protein activity level in a cell (e.g., a liver cell). Compounds thus identified can be used to treat conditions characterized by abnormal GADD45β expression or activity, e.g., cirrhosis and liver cancer.

The candidate compounds of the present invention can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann, et al. (1994) J. Med. Chem. 37, 2678-85; and Lam (1997) Anticancer Drug Des. 12, 145.

Examples of methods for the synthesis of molecular libraries can be found in the art, for example, in: DeWitt, et al. (1993) PNAS USA 90, 6909; Erb, et al. (1994) PNAS USA 91, 11422; Zuckermann, et al. (1994) J. Med. Chem. 37, 2678; Cho, et al. (1993) Science 261, 1303; Carrell, et al. (1994) Angew. Chem. Int. Ed. Engl. 33, 2059; Carell, et al. (1994) Angew. Chem. Int. Ed. Engl. 33, 2061; and Gallop, et al. (1994) J. Med. Chem. 37, 1233.

Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (Ladner, U.S. Pat. No. 5,223,409), spores (Ladner, U.S. Pat. No. 5,223,409), plasmids (Cull, et al. (1992) PNAS USA 89, 1865-1869), or phages (Scott and Smith (1990) Science 249, 386-390; Devlin (1990) Science 249, 404-406; Cwirla, et al. (1990) PNAS USA 87, 6378-6382; Felici (1991) J. Mol. Biol. 222, 301-310; and Ladner supra).

To identify compounds that modulate the GADD45β gene expression level or GADD45β protein activity level in a cell, a cell (e.g., a liver cell) is contacted with a candidate compound and the expression level of the GADD45β gene or the activity level of the GADD45β protein is evaluated relative to that in the absence of the candidate compound. The cell can be a cell that contains the GADD45β gene yet does not naturally expresses it, a cell that naturally expresses GADD45β, or a cell that is modified to express a recombinant nucleic acid, for example, having the GADD45β promoter fused to a marker gene. The cell can also be a cell treated with S-adenosylmethionine or an alcohol, a cell expressing a p53 gene at a level lower than that in a normal cell, a cell in which transcription of the GADD45β gene is directed by a promoter containing SEQ ID NO:1 and that expresses a CCAAT/NF-Y factor or an NF-κB factor, or a cell of a combination of just-described characteristics. The level of the GADD45β gene expression or the marker gene expression and the level of the GADD45β protein activity or the marker protein activity can be determined by methods described above and any other methods well known in the art. When the expression level of the GADD45β gene or the marker gene or the activity level of the GADD45β protein or the marker protein is higher in the presence of the candidate compound than that in the absence of the candidate compound, the candidate compound is identified as a potential drug for treating a liver disease.

This invention also provides a method for treating a liver disease. Subjects to be treated can be identified, for example, by determining the GADD45β gene expression level or the GADD45β protein level in a sample prepared from a subject by methods described above. If the GADD45β gene expression level or the GADD45β protein level is lower in the sample from the subject than that in a sample from a normal person, the subject is a candidate for treatment with an effective amount of compound that modulates the GADD45β level in the subject.

The treatment method can be performed in vivo or ex vivo, alone or in conjunction with other drugs or therapy.

In one in vivo approach, a therapeutic composition (e.g., a composition containing a compound that modulates the GADD45β gene expression level or the GADD45β protein activity level in a cell or a GADD45β protein itself) is administered to the subject. Generally, the compound will be suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. For treatment of a liver disease, the compound can be delivered directly to the liver tissue.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a polynucleotide containing a nucleic acid sequence encoding a GADD45β protein can be delivered to the subject, for example, by the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al. (1995) J. Mol. Med. 73, 479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the GADD45β protein is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., an increased GADD45β level) in a treated subject. As is well known in the medical arts, the dosage for any one subject depends upon many factors, including the subject's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

An ex vivo strategy for treating subjects with a liver disease associated with inadequate GADD45β activity can involve transfecting or transducing cells obtained from the subject with a polynucleotide encoding a GADD45β protein. Alternatively, a cell can be transfected in vitro with a vector designed to insert, by homologous recombination, a new, active promoter upstream of the transcription start site of the naturally occurring endogenous GADD45β gene in the cell's genome. Such methods, which “switch on” an otherwise largely silent gene, are well known in the art. After selection and expansion of a cell that expresses GADD45β at a desired level, the transfected or transduced cells are then returned to the subject. The cells can be any of a wide range of types including, without limitation, neral cells, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the GADD45β protein for as long as they survive in the subject.

The ex vivo methods include the steps of harvesting cells from a subject, culturing the cells, transducing them with an expression vector, and maintaining the cells under conditions suitable for expression of the GADD45β gene. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the GADD45β gene. The cells may then be injected or implanted into the subject.

Additionally, the therapeutic composition for treating a liver disease can contain S-adenosylmethionine, a nucleic acid encoding a p53 protein, or a p53 protein itself. These compositions and the GADD45β compositions described above can be used independently or as a combination.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

GADD45β Gene is Down-regulated in Human Cirrhosis and Liver Cancer

Global gene expression profiling with microarray was used to analyze changes of the expression levels of 12,800 genes and oligos in HCC (hepatocellular cancer) tissue and matching normal liver tissue. The microarray experiment was repeated 4 times, and the results were analyzed with different software. GADD45β was found to be one of the least expressed genes in hepatocellular carcinoma compared to normal liver.

Northern blot was conducted to confirm the microarray results. A 213-bp GADD45β cDNA probe, including GADD45β exon 3, was obtained by RT-PCR according to the GADD45β sequence of XM_{—}030487 in GenBank. GAPDH was used as a control for RNA loading. Compared to the expression in normal liver tissue, the expression of GADD45β was lower in liver cancer tissue and liver cancer cell line HepG2 and Hep3B.

Further, immunohistochemistry (IHC) study demonstrated that GADD45β is under-expressed in alcohol cirrhosis and liver cancer tissues than in normal liver tissue. The GADD45β expression in chronic cirrhosis tissue remains higher than in the matching foci of liver cancer tissue. In contrast, GADD45β is not under-expressed in colon cancer, breast cancer, prostate cancer, lymphoma, squamous carcinoma, or sarcoma tissue, suggesting that under-expression of GADD45β is liver tissue-specific.

Expression of human GADD45α and GADD45β mRNAs in different tissues was examined by Northern blot. GADD45β mRNA was readily detected in kidney, liver, and lung, suggesting that the GADD45β gene expresses in detoxifying tissues. GADD45α mRNA was detected in most tissues as GADD45β, but was found to be increased in skeletal muscle tissue. Both GADD45α and β have the highest expression level in normal liver tissue, suggesting that they are liver tissue-specific genes.

Expression of GADD45α and GADD45β after alcohol treatment was determined by Northern blot in HepG2 clone. It was revealed that the expression of GADD45α increases in a dose-dependent manner after stress from alcohol, yet the expression of GADD45β does not change. This result suggests that these two genes have different roles in alcohol-related DNA damage despite of 80% homology between these two gene family members. Lack of response of GADD45β to alcohol stress in HepG2 cells suggests that GADD45β dysfunction is associated with human hepatocarcinogenesis.

SAMe Induces GADD45β Expression

Induction of GADD45β by SAMe shows different response patterns in CL-48 normal liver cells and HepG2 cells. CL-48 and HepG2 cells were treated with 0, 1.0 mM, and 2.0 mM SAMe. After 72 hours of treatment, Northern blot was performed to analyze the expression of GADD45β, using 18S RNA as an internal loading control. The results demonstrate that GADD45β is induced by a low dose of SAMe in CL-48 cells, and the induction reaches plateau at a higher dose of SAMe. In HepG2 cells, however, the induction remains dose-dependent. This result suggests that SAMe induces apoptosis of liver cancer cells via GADD45β.

GADD45β Expression is p53-Dependent

Expression of GADD45β after SAMe treatment under different p53 backgrounds was examined by Northern blot. The expression of GADD45β in HepG2 (p53 wild type) was up-regulated by SAMe in different dose ranges. In contrast, in Hep3B (p53 null), expression of GADD45β failed to be induced by SAMe. This result suggests that the increased GADD45β expression induced by SAMe is p53-dependent.

Analysis of GADD45β Promoter

Luciferase activity directed by the GADD45β proximal promoter region was measured and normalized against the control β-Gal activity. A −870 through −1 bp fragment showed the highest promoter activity. The predicted promoter elements in this region include USF/N-Myc, NF-Y, CCAAT box and TATAA box.

Transcriptional Regulation of GADD45β by CCAAT/NF-Y Factor

Nuclear proteins were extracted from non-treated CL-48 cells, and from CL-48 cells 48 hours after treatment with 2.0 mM SAMe or 200 mM alcohol. End-labeled oligonucleotides corresponding to the CCAAT/NF-Y boxes of the GADD45β promoter region were incubated with the extracted nuclear proteins, and the binding complexes were then separated on a 6% retardation gel. Protein binding sites on the GADD45β promoter Pα region were identified using unlabeled oligonucleotide competitors and NF-Y antibodies. The amount of a major band decreased after treatment with SAMe compared to the non-treated controls. In the super-shift assay, the binding complex is shifted in SAMe-treated samples, consistent with GADD45β over-expression in SAMe-treated cells.

Transcriptional Regulation of GADD45β by NF-κB Factors

Nuclear proteins were extracted as mentioned above. End-labeled oligonucleotides corresponding to the NF-κB box of the GADD45β promoter region were incubated with the extracted nuclear proteins, and the binding complexes were then separated on a 6% retardation gel. Compared to non-treated CL-48 control cells, cells treated with SAMe or alcohol showed a decreased amount of one binding complex and an increased amount of another binding complex. This result indicates that transcription factors are involved in regulating the expression of GADD45β after administration of SAMe or alcohol.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of determining whether a subject is suffering from or at risk for developing a liver disease, the method comprising:
providing a sample from the subject, and
determining a GADD45β expression level in the sample, wherein the GADD45β expression level in the sample, if lower than that in a sample from a normal subject, indicates that the subject is suffering from or at risk for developing a liver disease.

2. The method of claim 1, wherein the sample is a liver tissue sample.

3. The method of claim 2, wherein the liver disease is cirrhosis.

4. The method of claim 2, wherein the liver disease is liver cancer.

5. A method of determining whether a subject is suffering from or at risk for developing a liver disease, the method comprising:
providing a sample from the subject, and
determining a GADD45β activity level in the sample, wherein the GADD45β activity level in the sample, if lower than that in a sample from a normal subject, indicates that the subject is suffering from or at risk for developing a liver disease.

6. The method of claim 5, wherein the sample is a liver tissue sample.

7. The method of claim 6, wherein the liver disease is cirrhosis.

8. The method of claim 6, wherein the liver disease is liver cancer.

9. A method of identifying a compound for treating a liver disease, the method comprising:
contacting a compound with a cell expressing a GADD45β gene, and
determining a GADD45β expression level in the cell, wherein the GADD45β expression level in the presence of the compound, if higher than that in the absence of the compound, indicates that the compound is a candidate for treating a liver disease.

10. The method of claim 9, wherein the cell is a liver cell.

11. The method of claim 10, wherein the liver disease is cirrhosis.

12. The method of claim 10, wherein the liver disease is liver cancer.

13. The method of claim 10, wherein the cell is treated with S-adenosylmethionine.

14. The method of claim 10, wherein the cell is treated with an alcohol.

15. The method of claim 10, wherein the cell expresses a p53 gene at a level lower than that in a normal cell.

16. The method of claim 10, wherein transcription of the GADD45β gene is directed by a promoter containing SEQ ID NO:1.

17. The method of claim 16, wherein the cell expresses a CCAAT/NF-Y factor or an NF-κB factor.

18. A method of identifying a compound for treating a liver disease, the method comprising:
contacting a compound with a cell expressing a GADD45β gene, and
determining a GADD45β activity level in the cell, wherein the GADD45β activity level in the presence of the compound, if higher than that in the absence of the compound, indicates that the compound is a candidate for treating a liver disease.

19. The method of claim 18, wherein the cell is a liver cell.

20. The method of claim 19, wherein the liver disease is cirrhosis.

21. The method of claim 19, wherein the liver disease is liver cancer.

22. The method of claim 19, wherein the cell is treated with S-adenosylmethionine.

23. The method of claim 19, wherein the cell is treated with an alcohol.

24. The method of claim 19, wherein the cell expresses a p53 gene at a level lower than that in a normal cell.

25. The method of claim 19, wherein transcription of the GADD45β gene is directed by a promoter containing SEQ ID NO:1.

26. The method of claim 25, wherein the cell expresses a CCAAT/NF-Y factor or an NF-κB factor.

27. A method of treating a liver disease, the method comprising:
identifying a subject suffering from or being at risk for developing a liver disease, and
administering to the subject a composition to increase a GADD45β level in the subject.

28. The method of claim 27, wherein the liver disease is cirrhosis.

29. The method of claim 27, wherein the liver disease is liver cancer.

30. The method of claim 27, wherein the composition is administered into a liver cell.

31. The method of claim 27, wherein the composition includes a nucleic acid encoding a GADD45β protein.

32. The method of claim 31, wherein the composition is administered into a liver cell.

33. The method of claim 27, wherein the composition includes a GADD45β protein.

34. The method of claim 33, wherein the composition is administered into a liver cell.

35. The method of claim 27, wherein the composition includes S-adenosylmethionine.

36. The method of claim 35, wherein the composition is administered into a liver cell.

37. The method of claim 27, wherein the composition includes a nucleic acid encoding a p53 protein.

38. The method of claim 37, wherein the composition is administered into a liver cell.

39. The method of claim 27, wherein the composition includes a p53 protein.

40. The method of claim 39, wherein the composition is administered into a liver cell.

41. A pharmaceutical composition comprising a nucleic acid encoding a GADD45β protein and a pharmaceutically acceptable carrier.

42. A pharmaceutical composition comprising a GADD45β protein and a pharmaceutically acceptable carrier.
